(19) United States
(12) Patent Application Publication
(10) Pub. No.: US 2006/0024248 A1
(43) Pub. Date: Feb. 2, 2006

(54) Composition and method employing membrane structured solid nanoparticles for enhanced delivery of oral care actives

(75) Inventors: Eric George Spengler, Ridgefield, CT (US); Gerd H. Dahms, Duisburg (DE)

Correspondence Address: FITZPATRICK CELLA HARPER & SCINTO, 30 ROCKEFELLER PLAZA, NEW YORK, NY, 10112, US

(73) Assignee: COMBE INCORPORATED, White Plains, NY (US)

(21) Appl. No.: 11/232,006

(22) Filed: Sep. 22, 2005

(30) Foreign Application Priority Data
Mar. 23, 2003 (WO) 2004/082666

(51) Int. Cl. A61K 8/18

(52) U.S. Cl. 424/49

(57) ABSTRACT

An oral care composition containing membrane-structured solid nanoparticles formed from a lyotropic liquid-crystalline mixed phase having an average particle diameter in the range of 10 to 1,000 nm. The nanoparticles are generally solid at 25° C. and are a combination of agent-carrier particles and emulsifiers. The membranes penetrate the nanoparticles so that the emulsifiers are present in the interior and on the surface of the nanoparticles.

### Cross-Reference to Related Applications

This application is a continuation-in-part application of International Patent Application Publication No. WO 2004/082666 and claims priority to this application under 35 U.S.C. § 120.

### BACKGROUND OF THE INVENTION

1. Field of the Invention

The present invention relates to an oral care composition comprising aqueous agent-carrier dispersion. In a particularly preferred embodiment, the present invention relates to an oral care composition useful in alleviating dry mouth, the composition comprising membrane-structured solid nanoparticles (MSSN) that are used as the agent-carrier.

2. Background of the Invention

Oral care products by which various oral care agents can be delivered to the soft and hard tissues of the oral cavity and throat have previously been known. Examples of such oral care products include, for example, products for relieving dry mouth symptoms, such as water, sugar free hard candy or gum, oral lubricants, such as Orajel Dry Mouth Moisturizing Gel™, Orajel Dry Mouth Moisturizing Spray™, Orajel Dry Mouth Moisturizing Toothpaste™, Bioténe Mouthwash™, Bioténe Dry Mouth Moisturizing Liquid™, prescription medicines, such as pilocarpine (Salagen™) or cevimeline (Evoxac™) and artificial or saliva substitutes such as Salivart™ or Mouth Kote™. Other examples of such oral care products include brushing aids such as dentifrice products for delivery of anti-caries actives such as fluoride or other actives for the reduction of the bacteria that lead to the formation of plaque, mouthwashes containing breath freshening actives and/or anti-bacterial actives, gels for treating tooth and gum pain, aerosols for treating sore throat pain, flexible strips containing breath freshening actives, and lozenges containing breath freshening actives and sore throat actives. Bleaching agents such as peroxide have also been developed that can be applied directly to the surfaces of the teeth, i.e., to the tooth enamel.

In the oral cavity, saliva is produced in the salivary glands and secreted into the mouth upon stimulation of the tissues of the oral cavity. Saliva has many functions, which include providing lubrication, limiting bacterial growth that can cause tooth decay and oral infections, promoting food digestion, and acting as a protective barrier against the demineralization of tooth enamel. Fluids, proteins, enzymes, and electrolytes are all found in saliva.

A condition in which the salivary glands do not secrete sufficient quantities of saliva, is commonly known as xerostomia or dry mouth. Xerostomia prevents adequate lubrication of the oral cavity, which results in oral cavity discomfort and difficulty in speaking and swallowing. Without saliva, the mouth, throat and tongue become dry and painful. In some instances, severe cracking of the tongue can result, increasing the possibility of infection. Teeth can also decay rapidly. Individuals suffering from xerostomia also may experience taste abnormalities, due to electrolyte imbalance.

There are several known causes of xerostomia. For example, xerostomia may result as an unwanted side effect of taking prescription medications. Xerostomia may also occur during states of elevated stress, anxiety, depression, or with certain endocrine diseases such as hypothyroidism, during chemotherapy, and with auto-immune disorders such as Sjogren's syndrome. In addition, people who have suffered trauma to the neck region may also develop xerostomia due to injury to the salivary glands. Xerostomia is a common affliction among elderly people.

Typically, xerostomia has been treated by orally administering a compound to promote or supplement the production of saliva. These compounds increase the moisture, fluids, and enzymes in the oral cavity. For example, mild cases of xerostomia have been treated by stimulating the secretion of saliva by sucking on hard candy and throat lozenges and/or by increasing the consumption of fluids. Changes in diet may also be implemented as part of the treatment. For example, the avoidance of certain beverages and foods, such as alcohol, caffeine, sugar, acidic foods, and other mouth drying consumables. Various techniques for treating xerostomia are found in U.S. Pat. Nos. 4,997,654; 6,159,459; 6,200,551 and 6,656,920.

Delivery Systems

The active substances used in pharmaceutical, cosmetic and/or food products are frequently encapsulated in active-substance carriers. This permits the active-substance carrier to be adapted to the particular use and permits a suitable dosage and release of the active substance. In the past, solid lipid nanoparticles (SLN) have been employed for this purpose. SLN represent a vehicle system that is an alternative to emulsions and liposomes. The nanoparticles can contain hydrophilic or hydrophobic pharmaceutical active substances, and may be administered orally or parenterally.

Typically in the past, the preparation of nanoparticles is conducted conventionally by high-pressure homogenization. In so doing, the lipid used as a matrix is melted and a suitable active substance is dissolved or dispersed in the melt. The active-substance-containing melt may be dispersed in an aqueous surfactant solution at the same temperature, while stirring. The dispersion thus obtained is then homogenized in the hot state in a high-pressure homogenizer. For example, a piston-gap homogenizer may be used, at pressures in the range of about 200 to about 1500 bar. An emulsion is obtained whose lipid phase recrystallizes when cooled, to yield solid lipid nanoparticles.

For other known methods for making nanoparticles, a cold homogenization process is employed, in which the active substance is again introduced into a molten lipid phase. The resulting mixed phase is then cooled, and the solid is ground to a particle size in the range of about 50 to about 100 μm. The lipid particles obtained in this manner are then dispersed in a cold surfactant solution, and the resulting dispersion is then subjected to high-pressure-homogenization to obtain nanoparticles. The SLN obtained by these methods have a solid core of an active-substance carrier surrounded by an emulsifier layer.

A process for the preparation of SLN dispersions is described, for example, in EP-B-0,167,825. The lipid nanopellets described therein are used as a vehicle system for drugs intended for oral use. Using a high-speed stirrer, lipid nanopellets are prepared by dispersing molten lipid in water. The desired particle-size distribution is then adjusted by ultrasonic treatment. As a rule, the stirring is done at rotational speeds in the range of 20,000 rpm. The resulting particles have average particle diameters in the range of 100 to 1000 nm.

EP-B-0,605,497 describes drug carriers consisting of solid lipid particles or solid-lipid nanospheres. They are prepared by high-pressure homogenization or high-pressure dispersion at pressures of 500 to 1550 bar. High-pressure homogenization may be achieved using, for example, a gap homogenizer. As a rule, a preliminary dispersion is carried out with a rotor-stator disperser.

A similar process is described in U.S. Pat. No. 5,885,486, where colloidally divided solid lipid particles are prepared by high-pressure homogenization of a lipid melt with an aqueous phase. The working pressures are 500 bar or more.

A review of the use of solid lipid nanoparticles as carriers for pharmaceutical and cosmetic active substances is found in J. Microencapsulation, 1999, Vol. 16, No. 6, pages 751 to 767. In particular, a discussion of how vitamin E is introduced into SLN systems is provided, which leads to improved penetration and action of vitamin E into and on the skin.

In J. Cosmet. Sci. 52, pages 313 to 324, the occlusion effects of SLN are described. In particular, the effect of skin moistening is investigated. A SLN formulation containing 40% cetyl palmitate and 5% surfactant in water was made by high-speed stirring; see formulation CPe in Table I. An average particle diameter of 3 μm was found; see Table II.

### SUMMARY OF THE INVENTION

One of the objects of the present invention is to provide a process for the preparation of an oral care composition comprised of a solid-nanoparticle dispersion wherein the nanoparticles are highly loadable, permit the inclusion of a wide range of agent-carriers and emulsifiers, and which allow surface modifications. The process does not rely on the use of high-pressure homogenization to obtain nanoparticles. Moreover, the process results in the formation of membrane-structured solid nanoparticles (MSSN).

As used herein, an oral care agent is any agent that has a desired effect in the oral cavity, including, inter alia, dry mouth-alleviating properties, and oral care (gingivitis, dry mouth, dental carries) treatments.

The present invention is an oral care composition comprising at least one oral care agent and membrane-structured solid nanoparticles (MSSN) having an average particle diameter in the range of 10 to 1,000 nm. The MSSN are preferably solid at 25° C. and are a combination of agent-carrier particles and emulsifiers. The MSSN comprise ultra thin layers or membranes which are formed from a lyotropic liquid-crystalline mixed phase. In one embodiment, the membranes comprise the entirety of the nanoparticles so that the emulsifiers are present in the interior and on the surface of the nanoparticles.

The present invention also provides a method of making the oral care composition having an oral care agent comprising the steps of:

(a) mixing an oral care agent-carrier at a temperature above the melting point or softening point of the oral care agent-carrier, to form a Phase B,

(b) mixing Phase B with a Phase A, at a temperature above the melting point or softening point of the oral care agent-carrier, wherein membranes are formed from a lyotropic liquid-crystalline mixed phase, and

(c) forming an aqueous oral care agent-carrier dispersion comprising oral care agent-carrier particles by combining the mixed phase with an aqueous Phase C, wherein the aqueous Phase C temperature is below the melting point or softening point of the oral care agent-carrier.

At least one emulsifying agent is added to Phase B and/or Phase A.

### BRIEF DESCRIPTION OF THE DRAWINGS

FIG. 1 is a graphical representation showing the variation of viscosity with the phase volume of the internal phase for an emulsion.

### DETAILED DESCRIPTION OF THE INVENTION

The oral care compositions of the present invention are comprised of membrane-structured solid nanoparticles (MSSN). The MSSN preferably have an average particle diameter in the range of 10 to 1,000 nm and are preferably solid at 25° C. The MSSN are prepared from agent-carrier particles and emulsifiers. In one embodiment, the membranes penetrate the entirety of the nanoparticles and comprise the structure of the nanoparticles so that the emulsifiers are present in the interior and on the surface of the nanoparticles.

The oral care MSSN of the invention exhibit improved bioadhesive properties, which provide for many advantages, including longer lasting lubrication and controlled released in a targeted manner.

In a preferred embodiment of the above-described method, the weight ratio of Phase B to Phase A is from about 40:1 to 1:40 and more preferably, from about 10:1 to 1:10. The Phase A may be any solution capable of forming membranes from a lyotropic liquid-crystalline mixed phase when mixed with Phase B. In one embodiment, Phase A is preferably an aqueous phase, such as, but not limited to, water, an aqueous solution, mixtures of water and surfactants with a hydrophilic-lipophilic balance (HLB) >7 and water-miscible liquids, or mixtures thereof. The Phase A may comprise an organic amphiphilic component such as Acesulfame Potassium, also known as Acesulfame K. In another embodiment, the Phase A may comprise an organic hydrophilic component, such as, but not limited to, polyhydric alcohols such as sorbitol, glycerine, xylitol, maltose and/or mannitol, and/or viscosity modifiers such as cellulosics, xanthan gum, and/or polyether.

Optionally, an emulsifier and/or co-emulsifier may be included in Phase A and/or Phase C. The present invention also contemplates adding an emulsifier to Phase C. The aqueous Phase C temperature may be, for example, at least 15° C. below the melting point or softening point of the agent-carrier. The oral care agent may be added to Phase B and/or Phase A.

Phase B includes an agent-carrier and may further include at least one emulsifying agent. Preferably a solid lipid is used as the agent-carrier. To assure a high degree of bioacceptance and good in vivo degradability, physiologically compatible lipids or lipids from physiological components, such as glycerides from endogenous fatty acids, are used. The preferred agent-carrier is a mixture of cetyl palmitate, bees wax and squalene. The agent-carrier and emulsifying agent are preferably mixed in a weight/weight ratio of about 200:1 to 10:1, and more preferably from about 20:1. The mixing temperature is preferably on the order of 55° C. to 75° C. for most agent-carriers.

Mixing is conducted such that Phase B and Phase A are blended together using lamellar flow.

Phase A is preferably an aqueous solution of cationic, amphoteric or nonionic surfactants or mixtures thereof. The weight/weight ratio of Phase B to Phase A is most preferably from about 3 to 1. The mixing temperature of Phase B and Phase A is most preferably from about 55° C. to 75° C. Phase C is preferably an aqueous solution of polyols, such as glycerol, sorbitol, xylitol and manitol. The weight/weight ratio of the liquid-crystalline mixed phase to Phase C is preferably about 90:1 to 1:90, more preferably about 5:1 to 1:5 and most preferably 8 to 2. The temperature of Phase C is most preferably 45° C.

It has been found according to the invention that aqueous agent-carrier dispersions containing lipid-based solid agent-carrier particles, having an average diameter preferably in the range of about 10 to about 1000 nm, and more preferably in the range of about 100 to about 400 nm, can be advantageously prepared if a Phase B (e.g., lipid melt) is mixed with a Phase A (e.g., an aqueous phase) heated to the same temperature in a weight ratio of about 10:1 to 1:10. In so doing, the mixture can be obtained without the use of high pressure homogenizers. In a preferred embodiment, the mixture can be obtained using a conventional mechanical stirrer having the mixing performance of a household mixer (or domestic kitchen mixer). In a laboratory operation, an adequate stirring effect could be obtained, e.g., using a Braun® kitchen mixer having a mixer head in the form of a two-bladed propeller having an overall diameter of 50 mm. In a preferred embodiment, the mixing propeller is surrounded by a protecting ring 63 mm in diameter. The maximum power consumption of the kitchen mixer is 350 W. A preferred mixer is model MR 550, Type 4189 manufactured by Benon.

The mechanical mixing in Step (b) and stirring in Step (c) is preferably carried out with mixers having a peripheral velocity in the range of about 0.1 to about 20 m/sec, and particularly preferably of about 1 to about 3 m/sec. The shearing effect of the mixer preferably corresponds to the shearing effect of a commercially available household stirrer or mixer, described above. By adhering to the Phase A to Phase B weight ratios, a very strong mixing effect can be achieved even at low shear-energy inputs.

In a preferred embodiment, the mixer may be a device such as the device described in International Patent Application Publication No. WO 2004/082817, the disclosure of which has been incorporated herein by reference.

Without being bound to any particular theory, it is believed that the lyotropic liquid-crystalline microemulsion obtained on mixing Phase B with the Phase A can be regarded as a system of two interpenetrating networks, so that the microemulsion exhibits a single-phase behavior and has a low shearing viscosity due to the small particle size. The weight ratio of Phase B to Phase A in Step (b) is more preferably about 2:1 to about 1:2, and most preferably about 1.5:1 to about 1:1.5.

Mixing phase B with phase A is preferably conducted under lamellar flow conditions, not turbulent flow conditions, to form minute droplets in which a liquid crystal structure is prevalent which is in the form of a high viscous emulsion. When the mixed phase is combined with an aqueous phase C, the gel rapidly forms droplets of membrane-structured solid lipid nanoparticles in which an agent is stored throughout the entire particle and the emulsifiers are present throughout the nanoparticles.

The MSSN are a combination of oral care agent-carrier particles and emulsifiers. In a preferred embodiment, the membranes are present throughout the nanoparticles so that the emulsifiers are present in the interior and on the surface of the nanoparticles.

Preferably, there are substantially no regions without membrane structure over the entire cross-section of the nanoparticles. The membranes are formed under lamellar flow conditions, preferably from a lyotropic liquid-crystalline mixed phase which, itself, preferably has an emulsifying action in the presence of water.

As noted above, in conventional SLN technology, an emulsifier layer surrounds a solid core of the agent-carrier. In contrast, the nanoparticles according to the invention also contain emulsifiers in the interior of the particles. Using the MSSN technology, all nanoparticles formed are preferably built-up from a membrane or membranes. Accordingly, the nanoparticles preferably have a uniform cross-sectional membrane-structure. Without being bound by any theory, it is believed that because the membrane layers constitute the particles, the MSSN have a significantly greater membrane surface area than SLN and can, therefore, carry more agents. In this way, it is possible, according to the invention, to introduce large amounts of agents into the nanoparticles.

International Patent Application Publication No. WO 2004/082666, the disclosure of which is incorporated herein by reference, describes a process for making membrane-structured SLN (MSSN) with an average size of 10 to 10,000 nm. The MSSN comprise a combination of agent-carrier particles and emulsifiers. Membranes penetrate the entire nanoparticle such that emulsifiers are present in the interior and on the surface of the nanoparticles. The process for preparing the MSSN does not require the use of costly high-pressure homogenizers but rather utilizes low cost mechanical mixing procedures. MSSN having a mean diameter of 10 to 10,000 nm are obtained by mixing an agent with a lipid-based agent-carrier and at least one emulsifier at a temperature above the melting point or softening point of the agent-carrier to form a Phase B, mixing Phase B with an aqueous Phase A at a temperature above the melting point or softening point of the agent-carrier to form a lyotropic liquid-crystalline mixed phase and diluting the mixed phase with an aqueous phase at a temperature below the melting point or softening point of the agent-carrier to obtain a dispersion containing the MSSN.

For example, typically amounts of up to about 60% by weight of agents, calculated on the loaded nanoparticles, can be introduced. In so doing, agents are stored not only in the superficial region of the nanoparticles, but also throughout the entire particle. This permits a highly targeted release of agents, even over a prolonged period of time.

The membrane structuring can be achieved through known liquid-crystalline systems, such as lamellar, hexagonal or cubic liquid-crystalline systems.

The liquid-crystalline mixed phase is generally anisotropic and thus appears turbid or opaque, and, in contrast to a microemulsion, not clear.

In the presence of water, the membrane-structured or lyotropic liquid-crystalline mixed phase has self-emulsifying properties; that is, emulsification occurs spontaneously at the water interface. Even at a high lipid loading, the membrane-structured or lyotropic liquid-crystalline mixed phase is electrically conductive. Such electrical conductivity shows that there is an aqueous external phase. In the preparation by the above-described method, the mixed phase forms a liquid-crystalline gel state prior to the dilution with aqueous Phase C. The dispersions finally obtained are free-flowing even at relatively high weight loadings of the MSSN phase. For example, dispersions with up to 60% by weight of MSSN phase (calculated on the total dispersion) are free-flowing. Typically the viscosity of the dispersion is similar to that of a cream or paste. Thus, free-flowing dispersions containing, e.g., 40% to 60% by weight of MSSN phase, can be readily prepared. The maximum attainable degree of loading depends, among other things, on the melting point of the loaded substance (oral care agent). If the agent has a low melting point, high loading degrees can be attained, typically on the order of 60% by weight.

The agents are preferably used in an amount of 0.1 to 60% by weight, and particularly preferably 1 to 10% by weight, based on the weight of Phase B.

A major advantage of the MSSN of the invention is that the agents can be released in a targeted manner, making a delayed release product possible by controlling the characteristics of the dispersion. During preparation, both particle size and release behavior can be controlled. Because the oral care composition of the present invention is generally used in the oral cavity, which is at or around the temperature of the body, in a preferred embodiment, the nanoparticle of the oral care composition has a melting point above body temperature (i.e., a melting point above about 37° C.) so that it does not rapidly melt when it enters the oral cavity.

Conventional through-the-skin (TTS) carrier suspensions increase skin hydration and/or behave as skin permeation enhancers by penetrating into the gums and swelling the gums, thereby opening the pores and penetrating them. Similarly, the MSSN of the invention can be applied to the gums and penetrate the gums, permitting the penetration of the oral care agent to reduce transepidermal water.

The MSSN of the invention may be applied to the mucosa and gums in the oral cavity. The very small particle size of the MSSN nanoparticles and their extremely large surface area provides outstanding coverage and hence strong lubricating properties.

The oral care composition of the present invention may be made into various forms that facilitate delivery and application to the oral cavity. For example, the oral care composition may be in the form of a mouth wash, a liquid center-filled lozenge, a tablet, a gel, a spray, a cream, a lotion, a liniment, a paste, a capsule, a gum or a dissolvable film. A dissolvable film may comprise a polymer substrate, such as pectin, to which the oral care composition of the invention is applied.

Listed below, by way of nonlimiting examples, are agents, which may be used, e.g., in the free form, or in the salt, ester or ether form:

Aromatic substances, flavoring agents (flavor oils and flavor extracts) and essential oils, which are known to persons skilled in the art. In a particularly preferred embodiment, the flavoring agents include all types of mints, including peppermint, spearmint, wintergreen, menthol, in the form of flavor oils and flavor extracts, as well as citrus based flavors, cinnamon, essential oils, such as menthol, eucalyptol, thymol, camphor, menthyl, salicylate, also known as wintergreen, and phenol, etc.

Nonlimiting examples of essential oils that may preferably be included in the oral care composition of the present invention include, for example, members selected from the group consisting of a or β-pinene; α-campholenic aldehyde; α-citronellol; α-iso-amyl-cinnamic (e.g., amyl cinnamic aldehyde); α-pinene oxide; α-cinnamic terpinene; α-terpineol (e.g., 1-methyl-4-isopropyl-1-cyclohexen-8-ol); λ-terpinene; achillea; aldehyde C16 (pure); α-phellandrene; amyl cinnamic aldehyde; amyl salicylate; anethole; anise; aniseed; anisic aldehyde; basil; bay; benzyl acetate; benzyl alcohol; bergamont (e.g., monardia fistulosa); bitter orange peel; black pepper; borneol; calamus; camphor; cananga oil (e.g., java); cardamon; carnation (e.g., dianthus caryophyllus); carvacrol; carveol; cassia; castor; cedar (e.g., hinoki); cedarwood; chamomile; cineole; cinnamaldehyde; cinnamic alcohol; cinnamon; cis-pinane; citral (e.g., 3,7-dimethyl-2,6-octadienal); citronella; citronellal; citronellol dextro (e.g., 3-7-dimethyl-6-octen-1-ol); citronellol; citronellyl acetate; citronellyl nitrile; citrus unshiu; clary sage; clove (e.g., eugenia caryophyllus); clove bud; coriander; corn; cotton seed; d-dihydrocarvone; decyl aldehyde; diethyl phthalate; dihydroanethole; dihydrocarveol; dihydrolinalool; dihydromyrcene; dihydromyrcenol; dihydromyrcenyl acetate; dihydroterpineol; dimethyl salicylate; dimethyloctanal; dimethyloctanol; dimethyloctanyl acetate; diphenyl oxide; dipropylene glycol; d-limonene; d-pulegone; estragole; ethyl vanillin (e.g., 3-ethoxy-4-hydrobenzaldehyde); eucalyptol (e.g., cineole); eucalyptus citriodora; eucalyptus globulus; eucalyptus; eugenol (e.g., 2-methoxy-4-allyl phenol); evening primrose; fenchol; fennel; ferniol; fish; florazon (e.g., 4-ethyl-α,α-dimethyl-benzenepropanal); galaxolide; geraniol (e.g., 2-trans-3,7-dimethyl-2,6-octadien-8-ol); geraniol; geranium; geranyl acetate; geranyl nitrile; ginger; grapefruit; guaiacol; guaiacwood; gurjun balsam; heliotropin; herbanate (e.g., 3-(1-methyl-ethyl) bicyclo (2, 2, 1) hept-5-ene-2-carboxylic acid ethyl ester); hiba; hydroxycitronellal; i-carvone; i-methyl acetate; ionone; isobutyl quinoleine (e.g., 6-secondary butyl quinoline); isobornyl acetate; isobornyl methylether; isoeugenol; isolongifolene; jasmine; juniper berry; lavender; lemon grass; lemon; lime; limonene; linallol oxide; linallol; linalool; linalyl acetate; linseed; litsea cubeba; 1-methyl acetate; longifolene; mandarin; mentha; menthane hydroperoxide; menthol crystals; menthol laevo (e.g., 5-methyl-2-isopropyl cyclohexanol); menthol; menthone laevo (e.g., 4-isopropyl-1-methyl cyclohexan-3-one); methyl anthranilate; methyl cedryl ketone; methyl chavicol; methyl hexyl ether; methyl ionone; methyl salicylate; mineral; mint; musk ambrette; musk ketone; musk xylol; myrcene; nerol; neryl acetate; nonyl aldehyde; nutmeg (e.g., myristica fragrans); orange (e.g., citrus aurantium dulcis); orris (e.g., iris florentina) root; para-cymene; para-hydroxy phenyl butanone crystals (e.g., 4-(4-hydroxyphenyl)-2-butanone); passion palmarosa oil (e.g., cymbopogon martini); patchouli (e.g., pogostemon cablin); p-cymene; pennyroyal oil; pepper; peppermint (e.g., mentha piperita); perillaldehyde; petitgrain (e.g., citrus aurantium amara); phenyl ethyl alcohol; phenyl ethyl propionate; phenyl ethyl-2-methylbutyrate; pimento berry; pimento leaf; pinane hydroperoxide; pinanol; pine ester; pine needle; pine; pinene; piperonal; piperonyl acetate; piperonyl alcohol; plinol; plinyl acetate; pseudo ionone; rhodinol; rhodinyl acetate; rosalin; rose; rosemary (e.g., rosmarinus officinalis); ryu; sage; sandalwood (e.g., santalum album); sandenol; sassafras; sesame; soybean; spearmint; spice; spike lavender; spirantol; starflower; tangerine; tea seed; tea tree; terpenoid; terpineol; terpinolene; terpinyl acetate; tert-butylcyclohexyl acetate; tetrahydrolinalool; tetrahydrolinalyl acetate; tetrahydromyrcenol; thulasi; thyme; thymol; tomato; trans-2-hexenol; trans-anethole and metabolites thereof; turmeric; turpentine; vanillin (e.g., 4-hydroxy-3-methoxy benzaldehyde); vetiver; vitalizair; white cedar; white grapefruit; and wintergreen, and the like. Particularly preferred are peppermint and mint flavor oils.

Oral pain relievers/anesthetics such as benzocaine, lidocaine, tetracaine, butacaine sulfate, benzyl alcohol, hexylreorcinol, menthol, phenol, phenolate sodium, salicylic alcohol, dyclonine HCl, hexylresorcinols, aspirin, acetaminophen, and the like.

Agents for the prevention of caries, which include, for example, fluoride, stannous fluoride, sodium fluoride, monofluorophosphate (MFP), and the like.

Antigingivitis/antiplaque agents, such as triclosan, quaternary ammonium compounds (e.g., cetyl pyridinium chloride and domiphen bromide), essential oils (e.g., eucalyptol, menthol, menthyl salicylate and thymol), phenol, stannous fluoride, and zinc salts (e.g. zinc citrate), polydimethylsiloxine.

Agents for relieving dry mouth, including pilocarpine, cevimeline, flavor oils (e.g., mint, citrus, etc.), polyols (e.g. xylitol, manitol, sorbitol, maltitol, etc), gums, such as xanthan gum, cellulosics (e.g. sodium carboxymehylcellulose, hydroxyethylcellulose, etc), enzymes (e.g. glucose oxidase, lactoperoxidase, and lysozyme, etc.) mucopolysaccharides, glycomannin, and fruit acids (citric acid, apple/malic acid, tartaric acid, etc.).

Enzymes such as primary dried yeast, lysozyme, lactoferrin, glucose oxidase, lactoperoxidases, dextranases, oxidases, etc.

Desensitizers such as potassium nitrate, strontium nitrate, calcium oxalate 2-hydroxyethyl methacrylate, and the like.

Whitening agents such as carbamide peroxide, and perhydrol urea.

Antiviral agents such as acyclovir, famciclovir, penciclovir, valacyclovir and docosanol.

Antibiotics/antifungals, such as polymyxin B and neomycin, clindamycin, penicillin, ketoconazole, clotrimazole, miconazole, chlorhexidine, and the like.

Cough suppressants and anti-tussives, including camphor, menthol and eucalyptus oil.

Expectorants such as guaifenesin (glyceryl guaiacolate).

Demulscents such as pectin, gelatin, glycerin, linseed, tragacanth and marshmallow.

Anti-inflammatories such as hydrocortisone and prednisone.

Antioxidants such as EGCG (epigallocatechin gallate), ursolic acid, rosemary extract, grape seed extract, pine bark extract, co-enzyme Q-10, superoxide dismutase, lutein, lycopene, astaxathin, alpha lipoic acid, tocopherol, bioperene and the like

Vitamins such as tocopherol, retinal, ascorbic acid, vitamin D, vitamin B1, vitamin B2, vitamin B3, pro-B5, B12, folic acid, vitamin C, and salts/esters thereof.

Bronchodilators such as albuterol, terbutaline, theophylline, ephedrine, epinephrine, and the like.

Antihistamines such as diphenhydramine HCl/citrate, chlorpheniramine maleate, brompheniramine maleate, clemastin fumarate, doxylamine succinate, phenindamine tartrate, tripolidine HCl, thonzylamine HCl, pyrilamine maleate, and dexchlorpheniramine maleate.

Decongestants such as pseudoephedrine HCl and phenylpropanolamine HCl.

Herbal compounds such as ephedra, feverfew, parthenolide, chamomile, licorice and derivatives, slippery elm, grape seed extract, garlic, acidophilus, bee propolis, chlorophyll, alfalfa, cardamon Echinacea, myrrh, peppermint, rosemary, and sage.

Odor neutralizers such as zinc salts, chlorophyll, and the like.

In a particularly preferred embodiment, the MSSN of the present invention are useful in alleviating dry mouth and include peppermint oil as the oral care agent. In another particularly preferred embodiment, the MSSN are useful in reducing gingivitis and include cetylpyridium chloride as the agent. In a further particularly preferred embodiment, the MSSN have anesthetic properties and include menthol and/or benzocaine as the agent. Certain preferred embodiments with these features are described in the Examples.

In one embodiment of the invention, an adhesive component, such as a polymeric bioadhesive or a mucoadhesive, may be used to coat the surface of the MSSN. The adhesive component is useful, inter alia, for linking desired agents or the like molecules to the external surface of the MSSN, for further improving the bioadherence properties of the MSSN, or for controlling or retarding the release of the agent from the MSSN. Molecules that may be linked to the MSSN via the adhesive component are preferably agent. In a further embodiment, the agent provides a more immediate relief or treatment when administered to a user because it is more readily available on the surface of the MSSN.

Any suitable adhesive component may be used. Nonlimiting examples of an adhesive component of the invention include hydrophobically modified hydrocolloids, such as, but not limited to, polycarbophil. Other hydrocolloids include chitosan, and poly methyl methacrylate. Chitosan is a natural mildly cationic polysaccharide. The adhesive component is preferably amphiphilic. A preferred amphiphilic hydrocolloid is Noveon® AA-1 Polycarbophil, USP. The MSSN may be coated with the adhesive component by any procedure available to the art. In one embodiment, the amphiphilic hydrocolloid is typically first hydrated in Phase A and then blended with Phase B to form the nanoparticle, In this embodiment, the amphiphilic nature of the hydrocolloid will coat the surface of the nanoparticle.

The agent linked to the MSSN via the adhesive component may be an oral care agent or any substance that provides a desired characteristic. For example, the agent may be a substance that controls the life of the MSSN, i.e., either increases the life or decreases the life. The agent may also be a substance that increases the bioadherence properties of the MSSN.

The oral care MSSN dispersions of the invention are storage-stable and have very good flow properties. The solid form of the particles and the inclusion of agents within the particles protect the incorporated agents against oxidative degradation, since the entry of oxygen is greatly reduced.

Compared with SLN technology, the choice of useable surfactants and diverse structures of useable waxes and lipids is greatly enlarged using MSSN technology. In addition, surface modifications are facilitated. As previously mentioned, the MSSN can be produced at a low cost and are highly loadable. Their properties can be adapted to a broad spectrum of requirements, independently of the agent-carrier. For example, different agents can be introduced and specifically incorporated in the agent-carrier phase also via an alcoholic, e.g., ethanolic solution or phase.

It is possible to incorporate both amphiphilic and hydrophobic agents into the MSSN of the invention, because the membrane structures have both hydrophilic and hydrophobic regions.

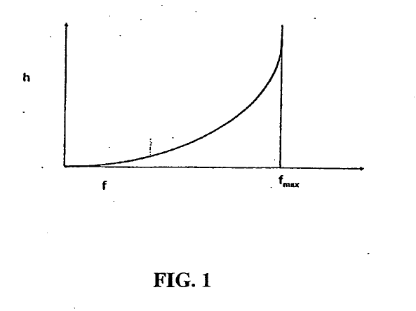

FIG. 1 shows the variation of the viscosity (h) with the phase volume (f) of the internal phase. The conventional preparation of processing emulsions is typically conducted below the maximum internal phase volume (fmax). In contrast, processing according to the present invention is carried out near or above (fmax) in a 3-phase process, so that a mixed lyotropic liquid-crystalline phase is obtained as an intermediate process step.

In conventional processes, a 2-step process produces the emulsion. However, in the present invention when the mixed lyotropic liquid-crystalline phase is combined with an aqueous phase, very small membrane particles are formed.

In a preferred embodiment the agent-carrier particles are lipid-based particles, including lipids and lipid-like structures. Examples of suitable lipids are the di- and triglycerides of saturated straight-chain fatty acids having 12 to 30 carbon atoms, such as unsaturated squalene, saturated squalene, lauric, myristic, palmitic, stearic, arachidic, behenic, lignoceric, cerotic, melesinic acid, as well as their esters with other saturated fatty alcohols having 4 to 22, preferably 12 to 22 carbon atoms such as lauryl, myristyl, cetyl, stearyl, arachidyl, behenyl alcohol, saturated wax alcohols having 24 to 30 carbon atoms such as lignoceryl, ceryl, cerotyl, myristyl alcohol. Preferred are di- and triglycerides, fatty alcohols, their esters or ethers, waxes, lipid peptides, branched chain fatty alcohols, or mixtures thereof. In particular, synthetic di- and triglycerides are used as single substances or in the form of a mixture, e.g., in the form of a hard fat. Examples of glyceryl tri-fatty acid esters are glyceryl trilaurate, glyceryl trimyristate, glyceryl tripalmitate, glyceryl tristearate or glyceryl tribehenate. Waxes are particularly preferred. Waxes that can be used according to the invention are natural waxes such as myristyl myristate, stearyl stearate, palmityl palmitate, behenyl behenate, solid ethers, like distearyl ether, dipalmityl ether, dibehenyl ether, plant waxes, animal waxes, mineral waxes and petrochemical waxes, chemically modified waxes such as hard waxes, and synthetic waxes. For a list of suitable waxes, reference may be made to Römpp Chemielexikon [Römpp's Chemical Encyclopedia], 9th edition, under the entry “Waxes” the disclosure of which is incorporated herein by reference. Suitable waxes are, e.g., beeswax, carnauba wax, candelilla wax, paraffin waxes, isoparaffin waxes, rice wax, cetyl palmitate and squaline. In a particularly preferred embodiment, beeswax is used. In another preferred embodiment, a mixture of cetyl palmitate and squalene is used. Further suitable waxes are, e.g., cetyl palmitate and cera alba (bleached wax, DAB [German Pharmacopoeia] 9). Suitable esters are derived, e.g., from branched-chain fatty acids and fatty alcohols, glycerol, sorbitan, propylene glycol, methyl glycoside, citric acid, tartaric acid, and maleic acid. Ceramide, phytosphingoside, cholesterol and phytosterols can also be used.

Additionally, carrier particles can be formed from polymers such as silicone waxes and PVP derivatives. These are, e.g., alkyl-substituted PVP derivatives, e.g., tricontanyl PVP, PVP-hexadecene copolymer, and PVP-eicosene copolymer. They can be used as vehicle materials, e.g., either alone or as admixtures to the lipids.

Liquid, semisolid and/or solid urethane derivatives can also be used, such as those marketed, e.g., by ALZO International Inc. They include, e.g., (branched) fatty alcohol dimer/IPDI, (linear) fatty alcohol dimer/IPDI, ethoxylated (branched) fatty alcohol dimer/IPDI, ethoxylated (linear) fatty alcohol dimer/IPDI, dimethiconol/IPDI copolymers, (hydrogenated) triglyceride ester/IPDI copolymers, ethoxylated (hydrogenated) triglyceride ester/IPDO copolymers, and aminated ethoxylated or nonethoxylated triglyceride ester/IPDI copolymers. Moreover, cross-linked polymers, polyacrylic acid derivatives, cellulose, xanthan gums and gum arabic can be used.

The amount of agent-carrier particles, based on the total aqueous agent-carrier dispersion, is preferably 0.1 to 30% by weight, and especially preferably 1 to 10% by weight.

Dispersion stabilizers may be used in addition to the lipids. They may be used, e.g., in amounts of 0.01 to 10% by weight, preferably 0.05 to 5% by weight. Broad, diverse classes of stabilizers can be employed including as anionic, cationic, amphoteric, and nonionic surfactants. Examples of suitable surfactants, include isethionates, diamide ether sulfates, alkyl polyglycosides, phosphoric acid esters, taurates, ethoxylated surbitan fatty acid esters, block polymers and block copolymers (such as, e.g., poloxamers and poloxamines), polyglycerol ethers and esters, lecithins of varied origin (e.g., egg or soy lecithin), chemically modified lecithins (e.g., hydrogenated lecithin), as well as phospholipids and sphingolipids, mixtures of lecithins with phospholipids, sterols (e.g., cholesterol and cholesterol derivatives, as well as stigmasterol), esters and ethers of sugars or sugar alcohols with fatty acids or fatty alcohols (e.g., sucrose monostearate), sterically stabilizing substances such as poloxamers and poloxamines (polyoxyethylene-polyoxypropylene block polymers), ethoxylated surbitan fatty acid esters, ethoxylated mono- and diglycerides, ethoxylated lipids and lipoids, ethoxylated fatty alcohols or fatty acids, and charge stabilizers or charge carriers such as, e.g., dicetyl phosphate, phosphotidyl glycerol and saturated and unsaturated fatty acids, sodium cholate, sodium glycocholate, sodium taurocholate or their mixtures, amino acids or peptizers such as sodium citrate (see J. S. Lucks, B. W. Müller, R. H. Müller, Int. J. Pharmaceutics 63, page 183 (1990)). Nonlimiting examples of cationic surfactants include cetylpyridinium chloride, cetylpyridinium bromide benzalkonium chloride, and benzethonium chloride. Nonlimiting examples of amphoteric surfactants include capryl/capramidopropyl betaine, and cocamidopropyl betaine. Nonlimiting examples of anionic surfactants include salts of acyl lactylates such as lauroyl lactylate, stearoyl lactylate, behenoyl lactylate, salts of acyl sulfates and their alkoxylated derivatives such as lauryl sulfate, cetyl sulfate, salts of diamide either sulfates, salts of akyl sarcosinates, such as lauroyl sarcosinate, salts of phosphoric fatty esters and their alkoxylated derivatives, such as lauryl phosphate, cetyl phosphate, isotridecyl phosphate, dicetyl phosphate, salts of alkyl sulfosuccinates and their alkoxylated derivatives, such as disodium laureth sulfosuccinate, salts of acyl glutamates such as lauroyl glutamate, salts of saturated or unsaturated or branch chain fatty acids such as sodium laurate, potassium stearate, sodium oleate, calcium stearate, TEA isostearate, citrate esters, cholates, such as sodium cholate, and sodium glycolate. All other enumerated surfactants represent nonionic stabilizers.

Viscosity-increasing substances may also be used, such as cellulose ethers and esters (e.g., methylcellulose, hydroxyethyl cellulose, hydroxypropyl cellulose, sodium carboxymethyl cellulose), polyvinyl derivatives such as polyvinyl alcohol, polyvinyl pyrrolidone, polyvinyl acetate, alginates, polyacrylates (e.g., carbopol), xanthans and pectins. In addition, as thickeners organic and inorganic hydrocolloids or mixtures of both may be used. In preferred embodiments, the organic hydrocolloid may be cellulose ethers and esters, (e.g. methyl cellulose, hydroxypropyl cellulose, sodium hydroxymethyl cellulose), cationic modified cellulose derivatives, polyglycol-polyamine resins (e.g., polyquart 81), polyvinyl derivatives such as polyvinyl alcohol, polyvinyl pyrrolidone, alginates, locust bean, carrageenan, glucomannan, xanthan gum, arabic gum, pectins, chitin, chitosan, polyacrylates, (e.g., carbopol), hydrophobic modified polyacrylates (e.g., pemulen), polyacrylamides, and hydroxypropyl guar. In other preferred embodiments, the inorganic hydrocolloids are betonite, organic treated bentonite, laponite, hectorite and veegum.

Suitable for use as Phase A, when it is an aqueous phase are water soluble and/or dispersible surfactants. Suitable for Phase C, are water, aqueous solutions, or mixtures of water with water-miscible liquids such as glycerol or polyethylene glycol. Other additional components include natural polysaccharides, as mannose, glucose, fructose, xylose, trehalose, polyhydric akoliobas mannitol, sorbitol, xylitol and other polyols such as polyethylene glycol, as well as electrolytes such as sodium chloride. These additional components may be used in an amount of from about 1 to about 30% by weight calculated on the Phase A and/or Phase C. Xylitol, as well as other polyhydric alcohols, enhances mouth feel and sweetness perception.

In addition, homopolysaccharides such as, for example, glucose, galactose, fructose, xylose, N-acetylglucosamine, and glucan (e.g., oat beta glucan) may also be included in the oral care composition to further improve the bioadhesive properties of the composition to the treated area of the oral cavity.

If desired, further viscosity-increasing substances or charge carriers can be used, as they are described in EP-B-0,605,497. As thickening agents, e.g., polysaccharides, polyalkyl acrylates, polyalkyl cyanoacrylates, polyalkyl vinyl pyrrolidones, acryl polymers, polylactic acids or polylactides may be used.

A broad range of emulsifiers or surfactants may be used for the preparation of the MSSN. In principle, any conventional surfactant may be used as part of a suitable combination. Preferably, nonionic, amphoteric, and/or cationic modified emulsifiers are used.

In one embodiment, pharmaceutically acceptable emulsifiers or emulsifiers which are approved for cosmetic or food use can be employed.

Moreover it is also possible, according to the invention, to modify the surface of the MSSN with the aid of surfactants. By concomitant use or subsequent introduction of anionic, cationic, amphoteric surfactants, or additional surfactants, an interfacial coating of such surfactants can be attained which leads to desired surface modifications. To stabilize or modify the interfaces, hydrocolloids may be included.

In the MSSN the emulsifier concentration is preferably a maximum of 5% by weight, and, particularly, preferably a maximum of 3% by weight of surfactant (calculated on the agent-carrier). The lower limit of the amount of surfactant is preferably about 0.05% by weight, depending on the ultimate use.

Suitable emulsifiers which form lyotropic liquid crystal (LC) structures or lamellar structures are, for example, natural or synthetic products. The use of surfactant mixtures is also possible. Examples of suitable emulsifiers are the physiological bile salts such as sodium cholate, sodium dehydrocholate, sodium deoxycholate, sodium glycocholate, sodium taurocholate. Animal or vegetable phospholipids such as lecithins with their hydrogenated forms, and polypeptides such as gelatin with its modified forms can also be used.

Further examples of suitable co-emulsifiers are glycerol esters, polyglycerol esters, sorbitan esters, sorbitol esters, fatty alcohols, propyleneglycol esters, alkylglucocide esters, sugar esters, lecithin, silicone copolymers, wool wax, and mixtures or derivatives thereof. Glycerol esters, polyglycerol esters, alkoxylates and fatty alcohols as well as isoalcohols may, e.g., be derived from ricinus fatty acid, 12-hydroxystearic acid, isostearic acid, oleic acid, linoleic acid, linolenic acid, stearic acid, myristic acid, lauric acid and capric acid. Also suitable, in addition to the aforementioned esters, are the succinates, amides or ethanolamides of fatty acids. As fatty acid alkoxylates, the ethoxylates, propoxylates or mixed ethoxylates/propoxylates, in particular, may be employed. Furthermore, silicone surfactants such as silicone polyols and silicone betaines may be used.

It is preferred, according to the invention, to use emulsifier systems which are mixtures of co-emulsifiers (gel network formers such as fatty alcohols, fatty acids, sorbitan esters, etc.) and special hydrophilic surfactants, and which are capable of forming micelles in water. Surfactants forming myelin structures at the interface with aqueous solutions include, e.g., polyglycerol 10-tricaprylate, polyglycerol 10-trilaurate, polyglycerol 2-oleate, sodium lauroyl lactylate, sodium cocoyl lactylate, glycerol cocoate citrate lactylate, mono and diglycerol fatty acid esters preferably of C6-C22, fatty acids, and propylene glycol fatty acid esters preferably of C6-C22 fatty acids.

Balanced complex emulsifiers, such as Biobase® EP and Ceralution® H can also be used.

The ratio of hydrophilic surfactant to co-emulsifier, which is optimal for the preparation of MSSN, is preferably higher than the optimum ratio for gel network formation.

Waxes/polymers/lipids and emulsifiers are preferably each used in a weight ratio of about 50:1 to about 2:1, preferably about 30:1 to about 15:1.

In a particularly preferred embodiment, the oral care composition is formulated for alleviating dry mouth. In this embodiment, peppermint or mint flavor oils preferably serve as the agents within the nanoparticles, and nonionic and cationic modified emulsifiers are used. The oral care composition may further include excipients such as artificial sweeteners (saccharin, neotame, acesulfame potassium, and/or sucralose), polyhydric alcohols (sorbitol, glycerine, xylitol, maltose, and/or mannitol), and/or viscosity modifiers (cellulosics, xanthan gum, and/or polyether 1).

A method of alleviating dry mouth is also contemplated. The method comprises the step of administering to a subject in need thereof, an effective amount of an oral care composition, preferably having mint flavor oil as an oral care agent, for an effective period of time.

Without wishing to be bound by any particular theory, it is believed that improved relief for dry mouth is provided by the lubricating “soft” waxy bioadhesive nanoparticle, which has an enhanced cationic property. By extending the release of the flavor oil, e.g., peppermint flavor oil, from the nanoparticle, it is believed to naturally help stimulate the secretion of saliva in the oral cavity.

The agent-carrier particles present in the aqueous dispersions of the invention preferably show microscopic doubly-refracting interfaces, which derive from a bilayer or multilayer of the emulsifiers forming the lamellar structures i.e. built-up layer structures as Langmuir-Blodgett (LB) structures or liquid-crystalline phases.

It is possible, by means of the emulsifiers, to form a unilamellar or multilamellar system or a lyotropic liquid-crystalline mixed phase.

Further components of the aqueous agent-carrier dispersions prepared according to the invention are described in EP-B-0,605,497, EP-B-0,167,825 and U.S. Pat. No. 5,885,486. For suitable stabilizing substances and charge stabilizers in particular, reference is made to EP-B-0,605,497, which is incorporated herein by reference.

Certain known methods of preparing nanoparticle dispersions require the use of hazardous halogenated organic solvents. See, e.g., U.S. Pat. Nos. 6,835,396 and 6,720,008. According to one embodiment of the invention, the agent-carrier dispersions are prepared without the use of halogenated organic solvents.

The invention is explained in greater detail by the following examples, which are illustrations of certain preferred embodiments.

### EXAMPLE 1

Table I, provided below, details the composition of five dry mouth formulations prepared in accordance with the present invention. Table II provided below details the composition of four additional oral care formulations prepared in accordance with the present invention, including an anti-gingivitis formulation and three oral anesthetic formulations, one of which includes a bioadhesive amphiphillic hydrocolloid.

The compositions listed in Table I and Table II were prepared by mixing the components listed for Phase A and heating them to 50° C., mixing the components listed for Phase B and heating them to 65° C.-70° C. and mixing the components listed for Phase C at room temperature. The heated Phase A and Phase B were then mixed together in a vessel under lamellar flow conditions, which created a lyotropic liquid-crystalline mixed phase (AB). The lyotropic liquid-crystalline mixed phase AB was then mixed with Phase C to form a dispersion and the mixture was allowed to cool to room temperature.

TABLE I

| | | | Composition #: | | | | |
|---|---|---|---|---|---|---|---|
| | | | #1 Anionic/ | #2 Cationic | #3 Cationic | #4 Cationic | #5 Cationic |
| Phase | Ingredient: | Ingredient Function | Nonionic [%] | Gel [%] | Gel [%] | Spray [%] | Spray [%] |
| A | Purified Water | water | 3.00 | 0.70 | 4.50 | 3.00 | 1.32 |
| | Sodium Lauryl Sulfate | emulsifier/stabilizer | 0.50 | 0.00 | 0.00 | 0.00 | 0.00 |
| | Cetyl trimethylammonium bromide | emulsifier/stabilizer | 0.00 | 0.50 | 0.35 | 0.67 | 0.25 |
| | PEG-100 stearate | emulsifier/stabilizer | 0.00 | 0.00 | 0.00 | 0.53 | 0.30 |
| | Xanthan Gum | mouthfeel/stabilizer | 0.10 | 0.00 | 0.00 | 0.00 | 0.00 |
| | Glycerin | humectant | 0.00 | 2.40 | 1.75 | 4.67 | 2.25 |
| | Acesulfame Potassium | artificial sweetener | 0.00 | 0.00 | 0.00 | 0.08 | 0.05 |
| | Cocamidopropyl Betaine | emulsifier/stabilizer | 0.00 | 0.00 | 0.00 | 0.00 | 0.69 |
| B | Squalene | lipid/wax carrier | 0.00 | 0.00 | 7.00 | 7.33 | 4.50 |
| | Bees wax | lipid/wax carrier | 0.00 | 0.00 | 4.65 | 4.89 | 3.00 |
| | Cetyl palmitate | lipid/wax carrier | 20.00 | 19.25 | 2.35 | 2.44 | 1.50 |
| | Sodium Stearoyl Lactylate | emulsifier/stabilizer | 0.50 | 0.00 | 0.00 | 0.00 | 0.00 |
| | Biobase RS (Glyceryl Stearate, | | | | | | |
| | Cetearyl Alcohol, Sodium Stearoyl | | | | | | |
| | Lactylate, Tocopherol) | emulsifier/stabilizer | 1.50 | 0.00 | 0.00 | 0.00 | 0.00 |
| | Peppermint Flavor | flavor | 1.00 | 1.00 | 0.70 | 0.75 | 1.00 |
| | Glyceryl Laurate | emulsifier/stabilizer | 0.00 | 0.00 | 0.00 | 0.67 | 0.35 |
| | Glyceryl Stearate | emulsifier/stabilizer | 0.00 | 0.95 | 0.70 | 0.00 | 0.00 |
| | Neotame | artificial sweetener | 0.00 | 0.00 | 0.00 | 0.02 | 0/03 |
| C | Purified Water | water | 66.92 | 74.40 | 76.67 | 64.95 | 74.79 |
| | Ethyl Alcohol | preservative | 5.50 | 0.00 | 0.00 | 0.00 | 0.00 |
| | d-Xylitol | mouthfeel/sweetener | 0.00 | 0.00 | 0.00 | 10.00 | 0.00 |
| | Sorbitol | mouthfeel/sweetener | 0.00 | 0.00 | 0.00 | 0.00 | 10.00 |
| | Sodium Saccharin | artificial sweetener | 0.10 | 0.10 | 0.10 | 0.00 | 0.00 |
| | Hydroxyethyl cellulose | mouthfeel/stabilizer | 0.00 | 0.00 | 1.23 | 0.00 | 0.00 |
| | Polyether-1 | mouthfeel/stabilizer | 0.00 | 0.70 | 0.00 | 0.00 | 0.00 |
| | Sodium Carboxymethylcellulose | mouthfeel/stabilizer | 0.88 | 0.00 | 0.00 | 0.00 | 0.00 |
| | Formula Totals: | | 100.00 | 100.00 | 100.00 | 100.00 | 100.00 |

TABLE II

| | | | Composition #: | | | |
|---|---|---|---|---|---|---|
| | | | #6 | #7 | #8 | #9 |
| | | Ingredient | Anti | Oral | Oral | Oral Anesthetic |
| Phase | Ingredient: | Function | Gingivitis | Anesthetic | Anesthetic | w/bioadhesive |
| A | Purified Water | Water | 3.17 | 3.40 | 3.40 | 3.55 |
| | Cetyl trimethylammonium | emulsifier/stabilizer | 0.20 | 0.25 | 0.25 | 0.10 |
| | bromide | | | | | |
| | Cetylpyridium chloride | Anitgingivitis Acitve | 0.10 | 0.00 | 0.00 | 0.00 |
| | Dyclonine HCL | Anesthetic Active | 0.00 | 0.10 | 0.00 | 0.10 |
| | PEG-100 stearate | emulsifier/stabilizer | 0.52 | 0.52 | 0.52 | 0.52 |
| | Glycerin | Humectant | 4.54 | 4.54 | 4.54 | 4.54 |
| | Acesulfame Potassium | artificial sweetener | 0.08 | 0.08 | 0.08 | 0.08 |
| | Polycarbophil | Bioadhesive Polymer | 0.00 | 0.00 | 0.00 | 0.05 |
| B | Squalene | lipid/wax carrier | 7.34 | 7.34 | 7.34 | 7.34 |
| | Bees wax | lipid/wax carrier | 4.90 | 4.90 | 4.90 | 4.90 |
| | Cetyl palmitate | lipid/wax carrier | 2.45 | 2.45 | 2.45 | 2.45 |
| | Peppermint Flavor | Flavor | 1.00 | 1.00 | 1.00 | 1.00 |
| | Menthol | Anesthetic Active | 0.00 | 0.20 | 0.20 | 0.20 |
| | Benzocaine | Anesthetic Active | 0.00 | 0.00 | 5.00 | 0.00 |
| | Glyceryl Laurate | emulsifier/stabilizer | 0.67 | 0.67 | 0.67 | 0.67 |
| | Neotame | artificial sweetener | 0.03 | 0.03 | 0.03 | 0.03 |
| C | Purified Water | Water | 65.00 | 64.52 | 59.62 | 64.44 |
| | Sorbitol | mouthfeel/sweetener | 10.00 | 10.00 | 10.00 | 10.00 |
| | Sodium Hydroxide | pH Modifier | 0.00 | 0.00 | 0.00 | 0.03 |
| | Formula Totals: | | 100.00 | 100.00 | 100.00 | 100.00 |

### EXAMPLE 2

The dry mouth compositions of Table I were tested in a classic expert texture and flavor analysis panel to characterize product attributes. The compositions were evaluated versus a known-in-market benchmark, Orajel Dry Mouth Moisturizing Gel™.

The dry mouth (DM) panel consisted of approximately 12 trained expert graders (healthy volunteers) who were qualified to grade the intensity of four key texture properties (mouth coating, mouth moistness, stickiness and lubricity/slip) and three core flavor properties (aromatics, i. e. mint; basic taste, i.e. sweet/bitter; and chemical feeling factors such as cooling). Graders used a ten point attribute intensity scale where zero represented little or no evidence of the attribute and where ten represented an intense sensory response to the attribute being graded. A dose of approximately 0.75 gm (about equivalent to in-market instructions) was administered to each grader and responses were recorded initially, then at 2 minutes, 5 minutes, 10 minutes and 15 minutes. Because the graders were healthy subjects, to simulate dry mouth, crackers were used to artificially dry the mouth. Given the healthy state of the graders, salivary activity returns to normal quickly and in almost all cases by 15 minutes. Thus, compositions were not evaluated in this model for true long term properties. Rather, performance was graded relative to a known-in-market benchmark. The data are shown in Table III below.

TABLE III

| | | | | |
|---|---|---|---|---|
| Immediate | 2 min | 5 min | 10 min | 15 min |

TEXTURE
Mouth Coating

| | | | | | |
|---|---|---|---|---|---|
| Test 1 | | | | | |
| OraJel gel | 7.0 | 2.0 | 0.0 | 0.0 | 0.0 |
| Compsoition 1 | 4.0 | 1.5 | 0.8 | 0.5 | 0.0 |
| Test 2 | | | | | |
| OraJel gel | 5.0 | 0.0 | 0.0 | 0.0 | 0.0 |
| Composition 2 | 7.0 | 2.0 | 1.0 | 0.0 | 0.0 |
| Test 3 | | | | | |
| OraJel gel | 8.0 | 0.0 | 0.0 | 0.0 | 0.0 |
| Composition 3 | 4.0 | 1.0 | 0.0 | 0.0 | 0.0 |
| Test 4 | | | | | |
| OraJel gel | 4.5 | 1.0 | 0.0 | 0.0 | 0.0 |
| OraJel spray | 1.0 | 0.0 | 0.0 | 0.0 | 0.0 |
| Composition 4 | 1.3 | 0.0 | 0.0 | 0.0 | 0.0 |

Slip/Lubricity

| | | | | | |
|---|---|---|---|---|---|
| Test 1 | | | | | |
| OraJel gel | 9.0 | 7.5 | 5.5 | 6.0 | 5.0 |
| Compsoition 1 | 6.0 | 5.5 | 6.0 | 5.0 | 4.4 |
| Test 2 | | | | | |
| OraJel gel | 10.0 | 6.5 | 5.5 | 5.0 | 5.0 |
| Composition 2 | 7.0 | 6.0 | 5.0 | 5.0 | 5.0 |
| Test 3 | | | | | |
| OraJel gel | 7.5 | 4.0 | 4.0 | 5.0 | 5.0 |
| Composition 3 | 8.0 | 6.0 | 5.0 | 5.0 | 5.0 |
| Test 4 | | | | | |
| OraJel gel | 10.0 | 6.0 | 5.0 | 5.0 | 5.0 |
| OraJel spray | 5.0 | 5.0 | 5.0 | 5.0 | 5.0 |
| Composition 4 | 7.0 | 7.0 | 5.5 | 5.0 | 5.0 |

Mouth Moistness

| | | | | | |
|---|---|---|---|---|---|
| Test 1 | | | | | |
| OraJel gel | 5.0 | 6.5 | 4.5 | 4.5 | 4.5 |
| Compsoition 1 | 8.0 | 6.0 | 6.0 | 5.0 | 5.0 |
| Test 2 | | | | | |
| OraJel gel | 5.0 | 4.0 | 5.0 | 5.5 | 5.5 |
| Composition 2 | 9.0 | 7.0 | 6.0 | 6.0 | 5.0 |
| Test 3 | | | | | |
| OraJel gel | 5.0 | 4.2 | 3.8 | 4.0 | 4.5 |
| Composition 3 | 6.0 | 5.5 | 5.0 | 5.3 | 5.5 |
| Test 4 | | | | | |
| OraJel gel | 5.0 | 5.0 | 5.0 | 5.0 | 5.0 |
| OraJel spray | 5.0 | 5.0 | 5.0 | 5.0 | 5.0 |
| Composition 4 | 7.0 | 7.0 | 6.0 | 5.3 | 5.0 |

FLAVOR
Mint Impact

| | | | | | |
|---|---|---|---|---|---|
| Test 1 | | | | | |
| OraJel gel | 9.0 | 5.0 | 2.0 | 1.3 | 1.0 |
| Composition 1 | 11.0 | 6.5 | 4.5 | 3.5 | 2.0 |
| Test 2 | | | | | |
| OraJel gel | 7.0 | 4.5 | 1.5 | 0.0 | 0.0 |
| Composition 2 | 9.0 | 6.0 | 5.0 | 3.0 | 3.0 |
| Test 3 | | | | | |
| OraJel gel | 6.5 | 4.0 | 2.0 | 0.5 | 0.0 |
| Composition 3 | 8.0 | 4.5 | 2.5 | 1.5 | 1.0 |
| Test 4 | | | | | |
| OraJel gel | 6.5 | 3.3 | 1.0 | 0.5 | 0.0 |
| OraJel spray | 5.0 | 3.0 | 1.0 | 0.0 | 0.0 |
| Composition 4 | 8.5 | 3.0 | 1.5 | 0.5 | 0.0 |

Cooling

| | | | | | |
|---|---|---|---|---|---|
| Test 1 | | | | | |
| OraJel gel | 6.0 | 4.0 | 5.0 | 1.0 | 0.0 |
| Composition 1 | 9.0 | 7.0 | 5.0 | 4.0 | 2.5 |
| Test 2 | | | | | |
| OraJel gel | 5.0 | 2.5 | 1.5 | 1.0 | 0.0 |
| Composition 2 | 10.0 | 8.0 | 6.0 | 4.0 | 2.5 |
| Test 3 | | | | | |
| OraJel gel | 4.0 | 2.5 | 1.0 | 0.0 | 0.0 |
| Composition 3 | 7.0 | 4.0 | 2.8 | 1.5 | 0.0 |
| Test 4 | | | | | |
| OraJel gel | 3.0 | 2.0 | 1.5 | 0.0 | 0.0 |
| OraJel spray | 2.5 | 1.8 | 0.0 | 0.0 | 0.0 |
| Composition 4 | 6.0 | 5.7 | 4.0 | 2.0 | 1.0 |

As can be seen from Table III, the compositions of the present invention were at least equal to the ORAJEL™ product for all capabilities tested. In addition, the compositions of the present invention were superior to the ORAJEL™ product for mouth moistness and cooling capabilities. The anionic//nonionic (#1) and cationic (#2) compositions had longer lasting mouth coating capabilities than ORAJEL™.

### EXAMPLE 3

A study was performed with nineteen adults suffering from dry mouth to compare the performance of an oral care composition of the invention (Composition 3 of Table I) with respect to an over-the-counter mouth gel product known as ORAJEL Dry Mouth Moisturizing Gel™.

The study participants were separated into two groups, Group A and Group B. Group A participants followed a protocol that required them to start with a one week wash out period, and then use the inventive composition for one week. This was followed with another one week wash out period, and then use of the ORAJEL™ dry mouth product for one week. Group B participants followed a protocol that required them to start with a one week wash out period, and then use the ORAJEL™ dry mouth product for one week. This was followed with another one week wash out period, and then use of the inventive composition for one week.

The results of the study showed that the oral care composition of the invention and ORAJEL™ dry mouth product performed comparably. Both products showed no evidence of oral irritation and no change in oral tissue color, glistening, and appearance of the lips and tongue. In addition, the inventive composition and ORAJEL™ dry mouth product were each found to produce visible improvements in dryness after one week of use among 68% of the study participants.

Further, the inventive composition performed better than ORAJEL™ dry mouth product in two significant aspects. First, the study participants found that the inventive composition left the mouth feeling moister than with the ORAJEL™ product. This was noticeable upon waking and during eating and speaking. Secondly, the inventive composition was found to produce results that lasted longer than the ORAJEL™ product. In fact, participants indicated that the inventive composition lasted about 42% longer than the ORAJEL™ product.

The invention includes any obvious modifications which are understood by those skilled in the art. The invention is not to be limited except as set forth in the following claims.

What is claimed is:

1. An oral care composition comprising: membrane-structured solid nanoparticles having an average particle diameter in the range of 10 to 1,000 nm, which are solid at 25° C. and include at least one oral care agent, agent-carrier particles and emulsifiers, wherein the solid nanoparticles comprise membranes formed from a lyotropic liquid-crystalline mixed phase combined with an aqueous phase.

2. The composition according to claim 1, wherein the membranes comprise the entirety of the nanoparticles such that the emulsifiers are present in the interior and on the surface of the nanoparticles.

3. The composition according to claim 2, wherein the membrane structure is present over a cross-section of the nanoparticles.

4. The composition according to claim 1, wherein the lyotropic liquid-crystalline mixed phase is self-emulsifying in the presence of water.

5. The composition according to claim 1, wherein the nanoparticles contain from about 0.1% to about 60% by weight of the oral care agent, based on the total weight of the nanoparticles.

6. The composition according to claim 1, wherein the oral agent is selected from the group consisting of dry mouth-alleviating agents, flavoring agents, pain relievers and anesthetics, caries prevention agents, antigingivitis/antiplaque agents, enzymes, desensitizers, whitening agents, antiviral agents, antibiotics, cough suppressants, expectorants, demulscents, anti-inflammatory agents, antioxidants, vitamins, bronchodilators, antihistamines, decongestants, herbals, odor neutralizers, oxidation-sensitive agents, and mixtures thereof.

7. The composition according to claim 6, wherein the dry mouth-alleviating agent is selected from the group consisting of flavor oils and flavor extracts

8. The composition according to claim 7, wherein the dry-mouth-alleviating agent is selected from the group consisting of mint flavor oil, mint flavor extract, fruit flavor oil, fruit flavor extract, citrus flavor oil, citrus flavor extract, cinnamon flavor oil and cinnamon flavor extract.

9. The composition according to claim 8, wherein the dry-mouth-alleviating agent is selected from the group consisting of mint flavor oil and mint flavor extract.

10. The composition according to claim 1, further comprising an adhesive component, wherein the adhesive component coats the surface of the membrane-structured solid nanoparticles.

11. The composition according to claim 10, wherein the adhesive component is selected from the group consisting of a bioadhesive and a mucoadhesive.

12. The composition according to claim 10, further comprising an agent linked to the adhesive.

13. A method of making an oral care composition comprising the steps of:
(a) mixing an agent-carrier, at a temperature above the melting point or softening point of the agent-carrier, to form a Phase B;
(b) mixing said Phase B and an Phase A, at a temperature above the melting point or softening point of the agent-carrier, to form membranes from a lyotropic liquid-crystalline mixed phase; and
(c) forming an aqueous agent-carrier dispersion by combining the mixed phase with an aqueous Phase C, wherein the aqueous Phase C is at a temperature below the melting point or softening point of the agent-carrier, and wherein at least one of Phases A, B or C includes at least one oral care agent,
wherein an emulsifying agent is present in at least Phase B or Phase A.

14. The method according to claim 13, wherein the oral care agent is present in Phase B.

15. The method according to claim 13, wherein the oral care agent is present in Phase A.

16. The method according to claim 13, wherein the oral care agent is present in Phase B and Phase A.

17. The method according to claim 13, wherein the agent-carrier dispersion has agent-carrier particles with an average diameter of about 10 to about 1,000 nm.

18. The method according to claim 13, wherein the Phase A is selected from the group consisting of water, water miscible liquids, and mixtures thereof.

19. The method according to claim 13, wherein a weight ratio of Phase B to Phase A is from about 40:1 to 1:40.

20. The method according to claim 13, wherein the agent is selected from the group consisting of dry mouth-alleviating agents, flavoring agents, pain relievers and anesthetics, caries prevention agents, antigingivitis/antiplaque agents, enzymes, desensitizers, whitening agents, antiviral agents, antibiotics, cough suppressants, expectorants, demulscents, anti-inflammatory agents, antioxidants, vitamins, bronchodilators, antihistamines, decongestants, herbals, odor neutralizers, oxidation-sensitive agents and mixtures thereof.

21. The composition according to claim 20, wherein the dry mouth-alleviating agent is selected from the group consisting of flavor oils and flavor extracts

22. The composition according to claim 21, wherein the dry-mouth-alleviating agent is selected from the group consisting of mint flavor oil, mint flavor extract, fruit flavor oil, fruit flavor extract, citrus flavor oil, citrus flavor extract, cinnamon flavor oil and cinnamon flavor extract.

23. The method according to claim 13, wherein the Phase A includes an emulsifier.

24. The method according to claim 13, wherein the aqueous Phase C includes an emulsifier.

25. The method according to claim 13, wherein the mixing in step (b) and the combining in step (c) are each carried out with a stirrer having a peripheral speed of about 0.1 to about 20 m/sec.

26. The method according to claim 13, wherein the weight ratio of the liquid-crystalline mixed phase to Phase C is about 90:1 to about 1:90.

27. The method according to claim 17, wherein the agent-carrier particles comprise diglycerides, triglycerides, fatty alcohols, their esters or ethers, waxes, lipid peptides or mixtures thereof.

28. The method according to claim 17, wherein the average diameter of the agent-carrier particles is about 100 to about 400 nm.

29. The method according to claim 13, further comprising a polyol or oil phase.

30. The method according to claim 17, further comprising the step of coating the agent-carrier particles with an adhesive component.

31. The method according to claim 30, wherein the adhesive component is selected from the group consisting of a bioadhesive and a mucoadhesive.

32. The method according to claim 30, further comprising the step of linking an agent to the adhesive component.

33. The method according to claim 13, wherein the Phase B comprises cetyl palmitate, sodium stearoyl lactylate, glyceryl stearate, cetearyl alcohol, tocopherol and peppermint flavor, wherein the Phase A comprises sodium lauryl sulfate, xanthan gum and purified water, and wherein the Phase C comprises purified water, ethyl alcohol, sodium saccharin and sodium carboxymethylcellulose.

34. The method according to claim 13 wherein the Phase B comprises cetyl palmitate, glyceryl stearate and peppermint flavor, wherein the Phase A comprises purified water cetyl trimethylammonium bromide and glycerin, and wherein the Phase C comprises purified water, sodium saccharin ethyl alcohol, sodium saccharin and polyether 1.

35. The method according to claim 13, wherein the Phase B comprises squalene, bees wax, cetyl palmitate, glyceryl stearate and peppermint flavor, wherein the Phase A comprises purified water, cetyl trimethylammonium bromide and glycerin, and wherein the Phase C comprises purified water, sodium saccharin and hydroxyethyl cellulose.

36. The method according to claim 13, wherein the Phase B comprises squalane, bees wax, cetyl palmitate, glyceryl laurate, neotame and peppermint flavor, wherein the Phase A comprises purified water, cetyl trimethylammonium bromide, asulfame potassium and glycerin, and wherein the Phase C comprises purified water and d-xylitol.

37. The method according to claim 13, wherein the Phase B comprises squalane, bees wax, cetyl palmitate, neotame, and peppermint flavor, wherein the Phase A comprises purified water, cetyl trimethylammonium bromide, polyethylene glycol-100 stearate, cocamidopropyl betain, glycerin and acesulfame potassium, and wherein the Phase C comprises purified water, and sorbitol.

38. The method according to claim 13, wherein the Phase B comprises squalane, bees wax, cetyl palmitate, glyceryl laurate, neotame and peppermint flavor, wherein the Phase A comprises purified water, cetyl trimethylammonium bromide, polyethylene glycol-100 stearate, cocamidopropyl betaine, glycerin, and acesulfame potassium, and wherein the Phase C comprises purified water and sorbitol.

39. The method according to claim 13, wherein the Phase B comprises squalane, bees wax, cetyl palmitate, menthol, glyceryl laurate, neotame and peppermint flavor, wherein the Phase A comprises purified water, cetyl trimethylammonium bromide, polyethylene glycol-100 stearate, dyclonine HCL, glycerine and acesulfame potassium, and wherein the Phase C comprises purified water, and sorbitol.

40. The method according to claim 13, wherein the Phase B comprises squalane, bees wax, cetyl palmitate, menthol, benzocaine, glyceryl laurate, neotame and peppermint, wherein the Phase A comprises purified water, cetyl trimethylammonium bromide, polyethylene glycol-100 stearate, glycerin and acesulfame potassium, and wherein the Phase C comprises purified water, and sorbitol.

41. The method according to claim 13, wherein the Phase B comprises squalane, bees wax, cetyl palmitate, menthol, glyceryl laurate, neotame and peppermint, wherein the Phase A comprises purified water, cetyl trimethylammonium bromide, dyclonine HCl, polyethylene glycol-100 stearate, glycerin, polycarbophil and acesulfame potassium, and wherein the Phase C comprises purified water, sodium hydroxide and sorbitol.

42. An oral care composition comprising membrane-structured solid nanoparticles formed from a lyotropic liquid-crystalline mixed phase containing a dry mouth-alleviating agent.

43. The oral care composition according to claim 42, wherein the dry mouth-alleviating agent is selected from the group consisting of peppermint oil or mint flavor oil.

44. The oral care composition according to claim 43, membranes comprise the nanoparticles so that the emulsifiers are present in the interior and on the surface of the nanoparticles.

45. The composition according to claim 42, wherein a membrane structure is present over a cross-section of the nanoparticles.

46. The composition according to claim 42, wherein lyotropic liquid-crystalline mixed phase is self-emulsifying in the presence of water.

47. The composition according to claim 42, wherein the composition further comprises at least one excipient selected from the group consisting of artificial sweeteners, polyhydric alcohols, and viscosity modifiers.

48. The composition according to claim 47, wherein the excipient improves the feel of the composition when administered to a mouth of a subject.

49. The composition according to claim 48, wherein the excipient is selected from the group consisting of xylitol, sorbitol, maltitol, lactitol, mannitol, glycerin, and artificial sweeteners.

50. The composition according to claim 49, wherein the artificial sweetener is selected from the group consisting of neotame, asulfame potassium, sucralose and saccharin.

51. The composition according to claim 42 or 43, wherein the nanoparticles are loaded with the xerostomia-treating agent in an amount up to about 60% by weight, based on the weight of the loaded nanoparticles.

52. A method of alleviating dry mouth, comprising: administering to a subject in need thereof, an effective amount of the oral care composition of claim 1 or 42, for an effective period of time.

53. The method of claim 52, wherein the agent is peppermint or mint flavor oil.

54. The method of claim 53, wherein the oral care composition is administered to the oral/bucal cavity as a spray, liquid cream, paste, gel capsule, strip lozenge or gum.

55. The method of claim 13, wherein a weight ratio of Phase B to Phase A is from about 10:1 to 1:10.

56. The method of claim 13, wherein a weight ratio of Phase B to Phase A is from about 2:1 to 1:2.

57. The method of claim 13, wherein a weight ratio of the lyotropic liquid-crystalline mixed phase to Phase C is from 5:1 to 1:5.
